(19) United States
(12) Patent Application Publication
(10) Pub. No.: US 2021/0093234 A1
(43) Pub. Date: Apr. 1, 2021

(54) Mild Traumatic Brain Injury Diagnostic Immunochromatographic Microneedle Patch

(71) Applicants: Amalia S Parra, Albuquerque, NM (US); Justin T Baca, Albuquerque, NM (US); Christina Salas, Albuquerque, NM (US); Evelyn Dohme, Albuquerque, NM (US); Barry Wood, Milpitas, CA (US); Janette Yuseil Mendoza, Albuquerque, NM (US); Arjun Senthil, Albuquerque, NM (US)

(72) Inventors: Amalia S Parra, Albuquerque, NM (US); Justin T Baca, Albuquerque, NM (US); Christina Salas, Albuquerque, NM (US); Evelyn Dohme, Albuquerque, NM (US); Barry Wood, Milpitas, CA (US); Janette Yuseil Mendoza, Albuquerque, NM (US); Arjun Senthil, Albuquerque, NM (US)

(73) Assignee: STC.UNM, Albuquerque, NM (US)

(21) Appl. No.: 15/733,218

(22) PCT Filed: Dec. 11, 2018
(86) PCT No.: PCT/US18/64891

§ 371 Date: Jun. 11, 2020

Related U.S. Application Data
(60) Provisional application No. 62/597,010, filed on Dec. 11, 2017.

(51) Int. Cl. A61B 5/145; A61B 10/00; A61B 5/00; G01N 33/558

(52) U.S. Cl. CPC A61B 5/14514; A61B 10/0045; G01N 2800/2871; G01N 33/558; A61B 2010/008; A61B 5/4064

(57) ABSTRACT

A diagnostic transdermal patch which utilizes a microneedle array and an integrated biochemical assay to detect the presence of biomolecules which are associated with a specific condition or disease, such as mild traumatic brain injury (MTBI).

### CROSS-REFERENCE TO RELATED APPLICATIONS

The following application claims benefit of U.S. Provisional Application No. 62/597,010, filed Dec. 11, 2017, which is hereby incorporated by reference in its entirety.

### BACKGROUND

Mild traumatic brain injury (MTBI), often called a concussion, is a common type of traumatic brain injury which is characterized by a mild blow to the head which results in short-lived neurological disturbances which typically resolve on their own, although serious complications can arise [1][2]. According to statistics presented by the Centers for Disease Control and Prevention, the incidence rate for MTBIs has increased substantially over the past decade and are predominantly caused by falls, traffic accidents, sports related accidents, and physical assaults [3]. Current MTBI diagnostic methods include psychological assessments which often rely on subjective, self-reported symptoms. Studies reviewing these types of assessments indicate that between 56% and 89% of patients who sustained an MTBI are incorrectly diagnosed [4].

### SUMMARY

The present disclosure provides a diagnostic transdermal patch which utilizes a microneedle array and an integrated biochemical assay to detect the presence of biomolecules which are associated with a specific condition or disease. According to a specific embodiment, the condition or disease may be mild traumatic brain injury (MTBI).

### BRIEF DESCRIPTION OF THE DRAWINGS

FIG. 1 is a top-view of a diagnostic patch according to an embodiment of the present disclosure.

FIG. 2 is a bottom-view of an embodiment of diagnostic patch according to the present disclosure.

FIG. 3 is a bottom-view of an alternate embodiment of a diagnostic patch according to the present disclosure.

FIG. 4 is a schematic illustration of the operation of the patch of FIG. 2.

FIG. 5 is a schematic illustration of the operation of the patch of FIG. 3.

FIG. 6 is a schematic illustration of an adhesive patch according to an embodiment of the present disclosure adhered to the skin 30 of a patient

### DETAILED DESCRIPTION

According to an embodiment the present disclosure provides a Mild traumatic brain injury (MTBI) diagnostic transdermal patch which utilizes an integrated biochemical assay to detect the presence of biomolecules which are associated with MTBI.

According to various embodiments, the patch detects and indicates the presence of an analyte in bodily fluid using, for example, immunochromatographic assays, otherwise known as lateral flow assays. However, unlike commonly known lateral flow assays such as off-the-shelf pregnancy and rapid HIV test, the MTBI diagnostic patch is applied to the skin for a short duration of time and utilizes a minimally-invasive microneedle array to draw interstitial fluid from a person suspected to have sustained an MTBI. The interstitial fluid is drawn through the patch via capillary action where it interacts with a series of bioactive molecules which bind to specific biomarkers which correlate with MTBI. The patch is easy to read and obviates the need for specialized personnel or equipment to interpret the results.

Microneedles are a minimally invasive way to obtain interstitial fluid which contain many biomarkers. These needles can be manufactured using materials such as, but not limited to, nickel, silica, and silicon carbide [12].

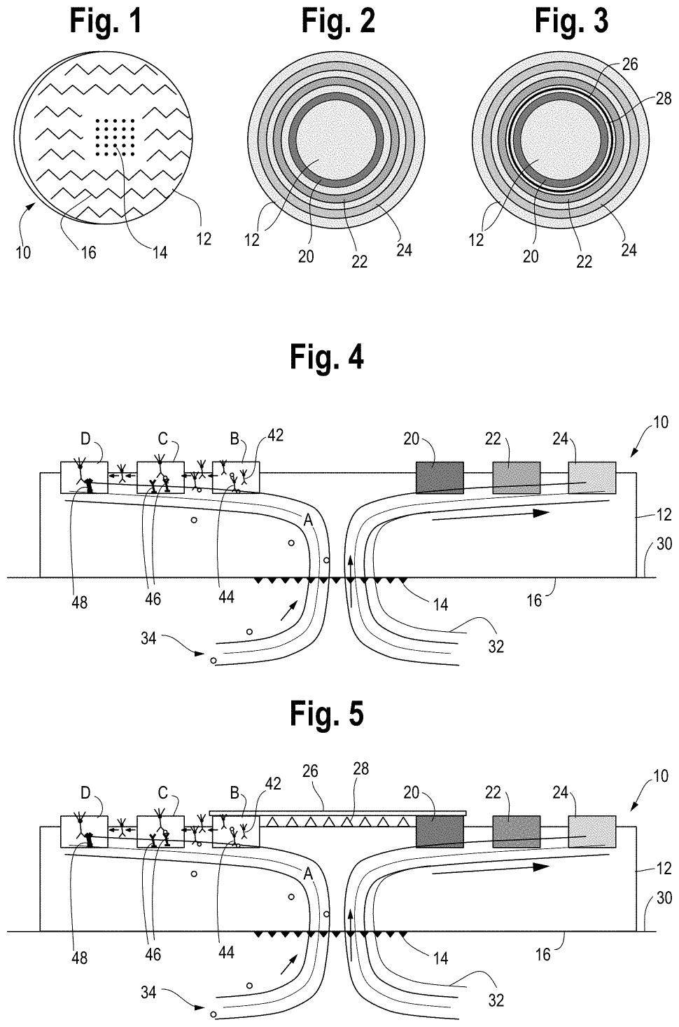

A specific embodiment of a diagnostic patch according to the present disclosure is shown in FIGS. 1-5. FIG. 1 shows a bottom view of an exemplary diagnostic patch 10. It should be understood that while the embodiment depicted in FIG. 2 shows the patch as being circular in shape, other shapes may also be used, as dictated by the particular design and intended use of the patch. In this embodiment, the base material of the patch comprises a substrate 12. Integrated into the center of the base material on the bottom surface is an array of absorbent microneedles 14. Also located on the bottom is a non-toxic and non-permanent adhesive 16.

In general, the substrate 12 should be a suitable substrate for the type of assay that is used by the diagnostic patch. For example, in an embodiment wherein the patch incorporates an immunochromatographic assay, the substrate could be an absorbent polymeric substrate.

For the purposes of the present disclosure, a “microneedle” is generally defined as a micromachined micron-sized structure that enables transport of a substance, such as interstitial fluid, though an interface, such as the dermal layer, via capillary action or other means. Microneedles are described, for example, in Miller, P. R. et al., (2018). Extraction and biomolecular analysis of dermal interstitial fluid collected with hollow microneedles. Communications biology, (1), 173; Romanyuk, A. V et al., (2014). Collection of analytes from microneedle patches. Analytical chemistry, 86(21), 10520-3; and Wu J. (2014) Microneedles: Applications and Devices. In: Li D. (eds) Encyclopedia of Microfluidics and Nanofluidics. Springer, Boston, Mass. According to some embodiments, the microneedles may be fabricated, for example, using a punch and die. Alternatively, commercially available microneedles may also be used or incorporated into the device.

In general, the adhesive 16 is capable of adhering the patch to the surface of the patient's skin long enough to draw interstitial fluid into the polymeric substrate and complete the immunochromatographic assay. Examples of suitable adhesives are described for example, in Cilurzo, F., et al., (2012). Adhesive properties: a critical issue in transdermal patch development. Expert opinion on drug delivery, 9(1), 33-45. Alternatively, the patch could be held in place with closure strips, surgical tape, or any other suitable means.

Turning now to FIG. 2, a top-view of the patch in FIG. 1 can be seen. As shown, in the depicted embodiment, the various diagnostic elements are concentric rings of materials deposited on the substrate 12. As shown, the rings of deposited materials are spaced apart such that they are interspersed with rings of unaltered substrate material. Moving outward from the center, the depicted embodiment includes, a film of soluble labeled antibodies (typically referred to in lateral flow assays as the “conjugate line”, or “conjugate ring” in the herein depicted embodiment showing a circular test) 20, a film of immobilized antibodies (typically referred to in lateral flow assays as the “test line,” or “test ring” as depicted herein), and a film of immobilized antigens (typically referred to in lateral flow assays as the “control line” or “control ring” as depicted herein). Of course it will be understood that other configurations for the various diagnostic elements are possible including linear arrangements, concentric squares (or other shapes), serpentine arrangements, etc.

While not shown in the figures, the patch may also include a barrier covering at least some of the bottom surface of the substrate to discourage, limit, or prevent reverse migration of bioactive reagents from the patch into the skin to which the patch is applied. This barrier may be, for example, an impermeable or controlled-directional flow membrane, film, or the like. Alternatively, the substrate itself may be designed to prevent flow of the reagents or sample back towards the skin.

FIG. 3 shows an alternative embodiment of the patch in FIG. 2, which includes an opaque cap 26 that covers the center and conjugate ring in order to prevent users from incorrectly interpreting the conjugate ring as the test ring, thereby reducing the number of false positive determinations made. According to a specific example of this embodiment, the opaque cap is secured to the patch via an adhesive 28, though other mechanisms for securing the cap may be used. Of course it will be understood that other mechanisms for obscuring the conjugate ring may be employed.

Existing research analyzing the biochemical changes following MTBI show there are significant changes in concentrations of certain biological molecules throughout the body. According to some embodiments, the analyte being detected should be a biomolecule that has relatively high detectable concentrations following MTBI when compared to baseline, variability between patients, and whose concentration is generally unaffected by external factors. There are myriad of biomolecules which have been identified as MTBI biomarker candidates which predominantly include proteins, messenger ribonucleic acids (mRNAs), micro-ribonucleic acid (miRNA) and have been detected in blood, cerebrospinal fluid (CSF), and saliva samples [4][7][8]. Differentially expressed proteins in MTBI include, but are not limited to, S100B, UCH-L1, GFAP, alpha-II spectrin, tau, and myelin basic protein (MBP) [10]. MiRNAs strongly associated with MTBI include, but are not limited to, miR-182-5p, miR-221-3p, mir-26b-5p, miR-320c, miR-29c-3p, miR-30e-5p, and miR 219-9 [9]. See also, Santa-Maria, I. et al., (2015). Dysregulation of microRNA-219 promotes neurodegeneration through post-transcriptional regulation of tau. The Journal of Clinical Investigation, 125 (2), 681-686; Bogoslovsky, T et al., (2016). Fluid biomarkers of traumatic brain injury and intended context of use. Diagnostics, 6 (4), 37. Kim et al., (2018) The current state of biomarkers of mild traumatic brain injury, JCL Insight v.3(1); Laskowitz D et al. (eds) (2016) “Translational Research in Traumatic Brain Injury” Chapter 12 Biomarkers of Traumatic Brain Injury and Their Relationship to Pathology, CRC Press/Taylor and Francis Group; Sharma et al., (2017) A blood-based biomarker panel to risk-stratify mild traumatic brain injury PLoS ONE 12(3); e013798; Agoston et al., (2017) Biofluid biomarkers of traumatic brain injury, Brain Injury, 31:9, 1195-1203, Martinez et al., MicroRNAs as diagnostic markers and therapeutic targets for traumatic brain injury, Neural Regen Res. (2017) 12(11), 1749-1761. Qin et al., (2018) Expression profile of plasma microRNAs and their roles in diagnosis of mild to severe traumatic brain injury, PloS ONE 13(9); e0204051; and Chandran et al., (2017) Differential expression of microRNAs in the brains of mice subjected to increasing grade of milk traumatic brain injury, Brain Injury, 31:1, 10-119.

It will be understood that while the present disclosure may make reference to the detection of “an analyte,” the patch may be designed to detect more than one analyte including, two or more, three or more, four or more, etc. Moreover, the patch may be designed to detect different types of analytes including, for example, one or more proteins and one or more MiRNAs.

It will be appreciated that while the present description is primarily directed to the detection of MTBI-associated analytes, the patch described herein could be used to test for or diagnose a nearly unlimited number of diseases or conditions simply by including a different or additional antibody, biosensor, or affinity component to the conjugate region, thus enabling the presently described patch to diagnose any number of conditions, diseases, etc. Examples of other analytes/conditions/diseases that might be detected using the patch described herein include, but are not limited to, Alzhemier's, multiple sclerosis, and Prion disease. See also, Ganesh, H. V., et al. (2016). Recent advances in biosensors for neurodegenerative disease detection. TrAC Trends in Analytical Chemistry, 79, 363-370. The presently described diagnostic patch would be of particular use when a patient is unable to describe or identify symptoms or provide other diagnostic criteria.

It will be understood that the antibody or antibodies used in the patch will be determined by the analyte being detected. Accordingly, in some embodiments it may be desirable to select analytes for which antibodies are already known and available or which can be easily obtained. Of course it will be understood that other types of affinity components other than antibodies could be used so long as there is a detectable interaction between the affinity component and the analyte being detected. This would enable other types of assays including, microfluidic ELISA, chemiluminescence immunoassays, hybridization assays, isothermal amplification, and the like.

FIGS. 4 and 5 show cross-sectional views of a patch 10 adhered to a patient's skin 30 and operation of the patch is best understood while viewing these Figures. For clarity, the rings on the right side of the figure are labeled to correspond with the description of the elements of the patch, while the left side of the figure are labeled to indicate the steps and processes that take place during use. As shown, at step A, once the patch 10 is adhered to the surface of the skin 30, an interstitial sample 32 possibly containing an analyte indicating MTBI 34 will begin to migrate up and radially outwards through the substrate 12 via capillary action. At step B, the sample will first encounter the inner annulus, or conjugate region 20, which comprises a film of soluble chemiluminescent or fluorescent coupled antibodies. If analyte is present in the sample, the analyte will bind to these labeled antibodies 42 forming a labeled analyte-antibody complex 44.

At step C, the sample will continue to migrate radially outwards to the middle annulus, or test region 22, which comprises a film of substrate bound antibodies 44. Labeled analyte-antibody complexes formed in the inner annulus will bind and aggregate in this area, producing a visible line. A visible line here would be a visual indication of the presence of analyte indicating MTBI in the sample. This would thus indicate that the subject has suffered a MTBI and a subsequence treatment protocol could be implemented.

At step D, the sample will continue to migrate radially outwards to the outer annulus 24, where unbound labeled antibodies will bind to substrate bound anti-antibodies and aggregate. Formation of a visible line on the outer annulus indicates that the interstitial fluid successfully passed through the test line region and that the test is complete.

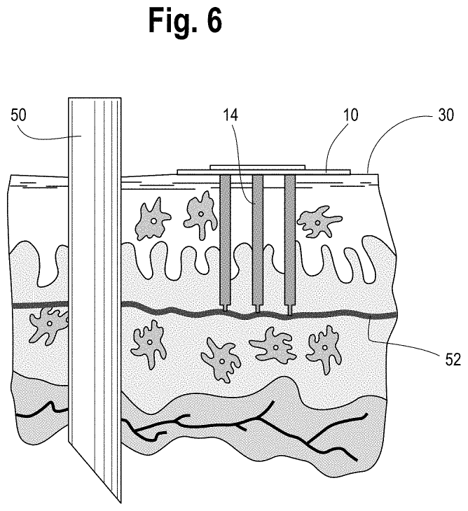

FIG. 6 is a schematic illustration of an adhesive patch according to an embodiment of the present disclosure adhered to the skin 30 of a patient. It can easily be seen that the microneedles 14 of the adhesive patch 10 are substantially less invasive than the hypodermic needle 50 and are able to sample interstitial fluid 52.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

References: All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.
[1] Mccrory, P., Meeuwisse, W. H., Aubry, M., Cantu, R. C., Dvoik, J., Echemendia, R. J., . . . . Turner, M. (2013). Consensus Statement on Concussion in Sport: The 4th International Conference on Concussion in Sport, Zurich, November 2012. Journal of Athletic Training, 48 (4), 554-575. doi:10.4085/1062-6050-48.4.05
[2] Vos, P. E., Alekseenko, Y., Battistin, L., Ehler, E., Gerstenbrand, F., Muresanu, D. F., . . . Wild, K. V. (2012). Mild traumatic brain injury. European Journal of Neurology, 19 (2), 191-198. doi:10.1111/j.1468-1331. 2011.03581.x
[3] Traumatic Brain Injury & Concussion. (2016, January 22). Retrieved Dec. 1, 2017, from https://www.cdc.gov/traumaticbraininjury/data/index.html
[4] Anto-Ocrah, M., Jones, C. M., Diacovo, D., & Bazarian, J. J. (2017). Blood-Based Biomarkers for the Identification of Sports-Related Concussion. Neurologic Clinics, 35 (3), 473-485. doi:10.1016/j.ncl.2017.03.008
[5] Ruan, S., Noyes, K., & Bazarian, J. J. (2009). The economic impact of 5-100B as a pre-head CT screening test on emergency department management of adult patients with mild traumatic brain injury. Journal of neurotrauma, 26 (10), 1655-1664.
[6]New Test Helps NFL Teams Detect Concussions. Retrieved Dec. 1, 2017, from http://abcnews.go.com/Sportss/story?id=99901 Roxhed, N. (2007).
[7] Mondello, S., Sorinola, A., Czeiter, E., Vimos, Z., Amrein, K., Synnot, A., . . . Buki, A. (2017). Blood-Based Protein Biomarkers for the Management of Traumatic Brain Injuries in Adults Presenting with Mild Head Injury to Emergency Departments: A Living Systematic Review and Meta-Analysis. Journal of Neurotrauma doi:10.1089/neu.2017.5182
[8] Overlapping MicroRNA Expression in Saliva and Cerebrospinal Fluid Accurately Identifies Pediatric Traumatic Brain Injury H., M., T., A., & ZIEMER, T. L. (1970, Jan. 26).
[9] Santa-Maria, I., Alaniz, M. E., Renwick, N., Cela, C., Fulga, T. A., Van Vactor, D., . . . Crary, J. F. (2015). Dysregulation of microRNA-219 promotes neurodegeneration through post-transcriptional regulation of tau. The Journal of Clinical Investigation, 125 (2), 681-686.
[10] Bogoslovsky, T., Gill, J., Jeromin, A., Davis, C., & Diaz-Arrastia, R. (2016). Fluid biomarkers of traumatic brain injury and intended context of use. Diagnostics, 6 (4), 37.
[11] Lahiji, S. F., Dangol, M., & Jung, H. (2015). A patchless dissolving microneedle delivery system enabling rapid and efficient transdermal drug delivery. Scientific reports, 5.
[12] Wang, M., Hu, L., & Xu, C. (2017). Recent advances in the design of polymeric microneedles for transdermal drug delivery and biosensing. Lab on a Chip, 17(8), 1373-1387. Chicago
[13]Wojnarowicz, M. W., Fisher, A. M., Minaeva, O., & Goldstein, L. E. (2017). Considerations for Experimental Animal Models of Concussion, Traumatic Brain Injury, and Chronic Traumatic Encephalopathy—These Matters Matter. Frontiers in Neurology, 8.
[14] Xiong, Y., Mahmood, A., & Chopp, M. (2013). Animal models of traumatic brain injury. Nature Reviews Neuroscience, 14 (2), 128-142.

What is claimed is:

1. A diagnostic patch comprising:
a microneedle array configured to obtain a sample of interstitial fluid from a subject; and
a lateral flow assay in fluid connection with the microneedle array wherein the lateral flow assay is configured to detect the presence of an analyte in the sample of interstitial fluid.

2. The diagnostic patch of claim 1 wherein presence of the analyte is indicative of mild traumatic brain injury (MTBI).

3. The diagnostic patch of claim 1 wherein the microneedle array and lateral flow assay are all contained within a substrate.

4. The diagnostic patch of claim 3 wherein the substrate provides the fluid connection between the microneedle array and the lateral flow assay.

5. The diagnostic patch of claim 4 wherein the substrate comprises a bottom surface that makes contact with the skin of the subject and comprises an adhesive configured to non-permanently adhere the patch to the subject's skin.

6. The diagnostic patch of claim 3 wherein the patch is round in shape and the microneedle array extends from the center of the bottom surface of the substrate.

7. The diagnostic patch of claim 6 wherein the lateral flow assay comprises concentric rings of diagnostic elements radiating outwards from the center of the substrate.

8. The diagnostic patch of claim 7 wherein the diagnostic elements comprise a conjugate region, a test region, and a control region.

9. The diagnostic patch of claim 8 wherein the conjugate region is obscured from view.

10. The diagnostic patch of claim 8 wherein the conjugate region comprises one or more antibodies which are biomarkers of MTBI.

12. The diagnostic patch of claim 10 wherein a biomarker is selected from the group consisting of: S100B, UCH-L1, GFAP, 1 alpha-II spectrin, tau, miR-182-5p, miR-221-3p, mir-26b-5p, miR-320c, miR-29c-3p, miR-30e-5p, miR 219-9, and myelin basic protein (MBP).

13. The diagnostic patch of claim 1 wherein the lateral flow assay comprises diagnostic elements comprising a conjugate region, a test region, and control region.

14. The diagnostic path of claim 13 wherein the conjugate region is obscured from view.

15. A method for detecting and/or diagnosing a disease or condition in a subject comprising placing a diagnostic patch on the subject wherein the diagnostic patch comprises:
a microneedle array configured to obtain a sample of interstitial fluid from a subject; and
a lateral flow assay in fluid connection with the microneedle array wherein the lateral flow assay is configured to detect the presence of an analyte indicative of the disease or condition in the sample of interstitial fluid.

16. The method of claim 15 wherein the microneedle array and lateral flow assay are all contained within a substrate.

17. The method of claim 15 wherein the disease or condition is mild traumatic brain injury.

18. The method of claim 15 wherein the analyte is selected form the group consisting of: S100B, UCH-L1, GFAP, 1 alpha-II spectrin, tau, miR-182-5p, miR-221-3p, mir-26b-5p, miR-320c, miR-29c-3p, miR-30e-5p, miR 219-9, and myelin basic protein (MBP).

19. The method of claim 15 wherein the lateral flow assay comprises a conjugate region, a test region, and a control region.

20. The method of claim 19 wherein the conjugate region is obscured from view.
